(19) United States
(12) Patent Application Publication
(10) Pub. No.: US 2004/0053395 A1
(43) Pub. Date: Mar. 18, 2004

(54) Determination of sample origin by a micro flora dna profiling method

(76) Inventors: Jagueline Horswell, Porirua (NZ); Stephen John Cordiner, Porirua (NZ); Elizabeth Wilhelmina Maas, Johnsonville (NZ); Kahurangi Bjorn, Wallace Sutherland, Porirua (NZ); Thomas Wilson Speir, Porirua (NZ); Tania Michelle Martin, Papakkowhai (NZ)

Correspondence Address: BANNER & WITCOFF, 1001 G STREET N W, SUITE 1100, WASHINGTON, DC, 20001, US

(21) Appl. No.: 10/416,151

(22) PCT Filed: Nov. 16, 2001
(86) PCT No.: PCT/NZ01/00257

(30) Foreign Application Priority Data
Nov. 17, 2000 (NZ) 508271

(51) Int. Cl. C12N 1/20

(52) U.S. Cl. 435/252.3

(57) ABSTRACT

The invention provides for a process suitable for determining the origin of one or more samples containing micro flora. The process involves the taking at least one sample of unknown origin, extracting the DNA from the micro flora of the sample, amplifying and conducting analysis of the amplified DNA, and comparing the analysis of the amplified DNA of the sample to that of a sample of known origin.

### FIELD OF THE INVENTION

The invention relates to a process for establishing the origin of a sample containing micro flora. In particular the present invention provides a process for making a comparison between the DNA profile of the micro flora within the sample of unknown origin and that of a sample of known origin.

### BACKGROUND OF THE INVENTION

It is often important, for example in forensic analysis, to make comparisons between physical evidence at a crime scene and physical evidence found in the presence or control of a suspect.

However, it has not been known previously to make a microbial DNA profile comparison between a sample of soil at a crime scene and a trace of soil in a suspect's possession or control. It is known that most soil matter contains a diverse micro flora community. Soil for example is a complex system consisting of, finely divided minerals and amorphous inorganic acids, animal, plant, pollens and microbial residues in various stages of decay, and a living, metabolising microbiota. This mixture is impacted by abiotic factors such as, topography, climate, parent materials, period of soil formation; and biotic factors such as macro and micro flora and human activity (Marumo et al. 1995; Thornton, 1986). Analysis of micro flora containing matter, such as soil, is seldom carried out in routine forensic examinations. In such context as a forensic study, matter origin analysis can play an important role in a criminal investigation as trace evidence, which can prove or disprove a link between a suspect and a crime scene.

Analysis of soil transferred from a crime scene has, for the most part, relied on abiotic factors. For example soil analysis is based on factors such as colour comparison, mineral examination and density gradient distribution (Marumo et al. 1995, Thornton, 1986). Soil and matter analysis based on abiotic factors is seldom carried out in routine forensic examinations. Principal reasons for this are; the range of skills needed and the years of experience for investigation are formidable; many techniques require a larger sample size than is likely to be encountered in many actual case situations (e.g.; soil in a shoe print); and in most cases the techniques applied by soil scientists are not easily applicable to the forensic purpose. (Marumo et al. 1995, Thornton, 1986). In New Zealand comparisons of pollen distributions in soils are often carried out to characterise soil samples (Horrocks and Walsh, 1998). Pollen analysis of this type is also difficult to achieve and evidence from pollen analysis studies has never been considered as providing sufficient stand alone evidence on which to argue a case. Difficulties are often encountered when presenting such evidence in court to a lay jury. The technical and scientific details are often difficult to interpret and describe convincingly in lay terms.

To date, analysis of soil microorganisms has been ignored by the forensic scientific community, largely because of the limitations of traditional culturing techniques. Only a small subset of organisms are amenable to isolation and characterisation (Bergquist et al., 1996; Yates, 1996; Ward et al., 1992).

The rapid growth of molecular technologies has allowed the use of polymorphic analysis to study the micro flora community dynamics within a soil sample. For example, the evaluation of terminal-restriction fragment length polymorphism is a possible tool for estimating the diversity within a soil community (Osborne et al., 2000). In a similar study the variable region of the 16 S-rRNA gene of Enterobacter colcae and Arthrobacter sp. in soil indicated that similar soil types contain similar dominating bacterial types. To date, the analysis of soil micro flora communities using polymorphic analysis has been directed towards the study of diverse environments, or the effect of pollution or environmental changes on such communities (Fisher and Triplett, 1999; Ward et al., 1992; Fournier et al., 1998; Leeflang and Smit, 1997; Moyer et al, 1996; Born man and Triplett, 1997). Although, these techniques have been applied to study the community dynamics within a soil micro flora population, there is no evidence that the micro flora diversity would be sufficient to allow this technique to be used to determine the origin of a sample of matter such as soil.

Accordingly, it is an object of this invention to overcome some of the limitations of existing micro flora sample analysis and provide a useful process for determining the origin of a sample containing micro flora or to at least provide the public with a useful alternative.

### SUMMARY OF THE INVENTION

This invention provides a process suitable for determining the origin of one or more samples containing micro flora, the process including the steps of:

taking at least one sample of unknown origin containing micro flora, and at least one sample of known origin containing micro flora,

extracting the DNA from the micro flora of each sample,

amplifying the DNA or any part thereof from each sample,

conducting an analysis of the amplified DNA for each sample, and

comparing the analysis of the amplified DNA from the sample of unknown origin with the sample of known origin.

Preferably, the process may be applied to a soil sample of unknown origin. Preferably the DNA to be amplified involves both a conserved and variable region of a gene, and most preferably the area amplified includes at least a portion of the 16S rRNA gene. It is preferred that the analysis of the amplified DNA is a polymorphic analysis, such as restriction fragment length polymorphism. Most preferably the polymorphism analysis may be performed on the 16S rRNA gene. Preferably the comparison of the polymorphic analysis from the samples is via a visual comparison or more preferably by a mathematical process. Preferably the process is applied to forensic analysis.

In a further aspect, this invention provides a suitable kit containing the materials required to amplify the suitable region of DNA, and perform the analysis defined above.

In applying the present invention, it has been suprisingly found that a polymorphic profile of the micro flora of a soil sample may be matched to a sample taken from the same location, while comparison to samples of different locations show considerable differences in the polymorphic profile.

Therefore, this invention may provide a useful tool in determining the origin of a sample containing micro flora, and may be particularly useful for, but not limited to, the area of forensic analysis. Further aspects of the invention will become apparent from the following description.

### BRIEF DESCRIPTION OF THE DRAWINGS

The invention will now be described by way of example only with reference to the accompanying drawings in which:

FIG. 1 shows a comparison of a microbial community profile for soil taken from a shoe print and a soil sample taken from a suspect's shoes.

FIG. 2 shows electropherograms of four reference soil samples of known origin.

### DETAILED DESCRIPTION OF THE INVENTION

This invention relates to a process of determining the origin of a sample containing micro flora by comparing the DNA profile of the micro flora with that of a sample of known origin or a database or library of standard micro flora profiles.

Samples containing micro flora that may be suitable for the process may include soil samples, silt samples, water samples, sediments, or soiled articles or clothing. The amount of sample required will depend on the type of sample to be analysed but would usually be <1 gm. It will be appreciated that the amount of sample should be of a sufficient size to ensure a representative population of the micro flora is included. For a quantitative analysis it is important that equal volumes or amounts of the sample(s) of unknown origin and that of the sample(s) of known origin are used.

Suitable DNA extraction techniques for extracting the total DNA from the various type of samples are well known, and the appropriate method would easily be determined by somebody skilled in the art.

In order to perform the polymorphic analysis, the DNA can be amplified by known methods, such as for example, the PCR method (polymerase chain reaction). The region of DNA to be amplified should be the same in both samples. Preferably the region would involve a primer directed towards a constant region, thereby ensuring that the largest population of micro flora DNA is amplified, while the area amplified includes a variable region, thereby allowing a broad polymorphic analysis. Suitable examples might, for example, include the 16S rRNA gene, 23S rRNA gene or the region between the 16S and 23S rRNA genes.

Any form of polymorphic analysis is suitable. However preferably the polymorphic analysis chosen would include multiple detectable products. It will be appreciated that the more variable products that are detectable the more determinate the analysis will be. This may be an important aspect if the results from this process are to be used as evidence in a court of law. For example, a restriction fragment length polymorphism analysis could be performed over the variable region of the 16S rRNA gene.

Comparison of the polymorphic analysis of the DNA from the sample of unknown origin to the DNA of the sample of known origin, can be achieved using a variety of methods. For example, for a restriction fragment length polymorphism analysis, comparison may be achieved by a visual comparison of an autoradiograph of a polyacrylamide gel electrophoresis, or alternatively the PCR product could be fluorescently-tagged and then laser detected and the electropherogram may be visually compared. Alternatively, the polymorphic profiles may be compared mathematically, such as for example using the Sorenson similarity index:

Cs=2NAB/NA+NB

Where

Cs=similarity index

NAB=number of matching peaks

NA=total number of peaks in soil A

NB=total number of peaks in soil B

Finally, it will be appreciated that the comparison may also be achieved by a suitable computer algorithm, which will include all suitable parameters. For example, for a restriction fragment length polymorphism analysis conducted using a fluorescently tagged PCR product wherein the products are detected using the GeneScan™ 3.1 software program (Applied Biosystems), suitable parameters might include both the presence and absence of peaks of a particular size as well as peak height and area.

Preferably, any such comparison would provide a simple index or figure giving a lay person an understanding of the degree of similarity between the samples or otherwise.

The invention also relates to a suitable kit to allow someone skilled in the art to perform the process outlined. Suitable components of such a kit would include the primers directed towards an appropriate polymorphic analysis, any materials required to perform the polymorphic analysis, and suitable materials for comparing sample results.

### EXAMPLE

Scenario 1—Footwear Impression

Reference soils (approximately 50 g wet weight) were collected from five different locations for analysis. (Table 1). At site A forensic samples were also collected. A shoe-print was made and soil recovered from the tread of the outsole (A1). Soil was also collected from the print at the scene (A2). Soil was homogenised by sieving to <2 mm and stored at 5° C. Sample identity was not made known to the analyst.

TABLE 1
Reference soil samples

| Site | Soil Type |
|---|---|
| A-forensic sample, soil | Clay loam |
| recovered from outside of shoe | |
| (A1), soil collected from shoe | |
| print (A2). | |
| B-reference sample | Silt loam |
| C-reference sample | Silty clay |
| D-reference sample | Fine sandy loam |
| E-reference sample | Clay |

High quality microbial nucleic acid suitable for Polymerase Chain Reaction (PCR) analysis was extracted from 200 mg of soil sample using Bio101198 protocol and “FastDNA kit for soil” with some modifications. Primers were chosen to amplify the DNA from the 16S ribosomal RNA of the bacteria. The 5′ end of the primers were labelled with a phosphoramidite dye and the amplified product was approximately 1300 base pairs. Following PCR, sample fragments were discriminated by using the ABI 310 genetic analyser (Applied Biosystems), in which DNA is electrophoresed in a capillary tube filled with electrophoresis polymer rather than polyacrylamide gel. The data was analysed using the GeneScan 3.1 software program (Applied Biosystems). Soil bacterial profiles were compared by determining the presence or absence of peaks and calculating the Sorenson similarity index:

Cs−2NAB/NA+NB

Where

Cs=similarity index

NAB—number of matching peaks

NA=total number of peaks in soil A

NB—total number of peaks in soil B

DNA fragments (peaks) between 75 bps and 490 bps and peak heights over 150 fluorescence units were included in the analysis. The 150 fluorescent unit minimum peak height was an arbitrary cut off point set prior to analysing the data.

For comparison, soil samples were also collected from four other sites.

Scenario 2—Soil Stained Clothing

An imprint was also made in the soil at another site by kneeling wearing a clean pair of denim jeans. Soil was sampled from the impressions made by each knee in the soil (left knee and right knee). The jeans were taken back to the laboratory and areas of soil staining removed for analysis. The left knee soil stain was extracted with water, the right knee stain was extracted with water plus Tris-EDTA (TE) buffer pH 8.0.

Results

Scenario 1

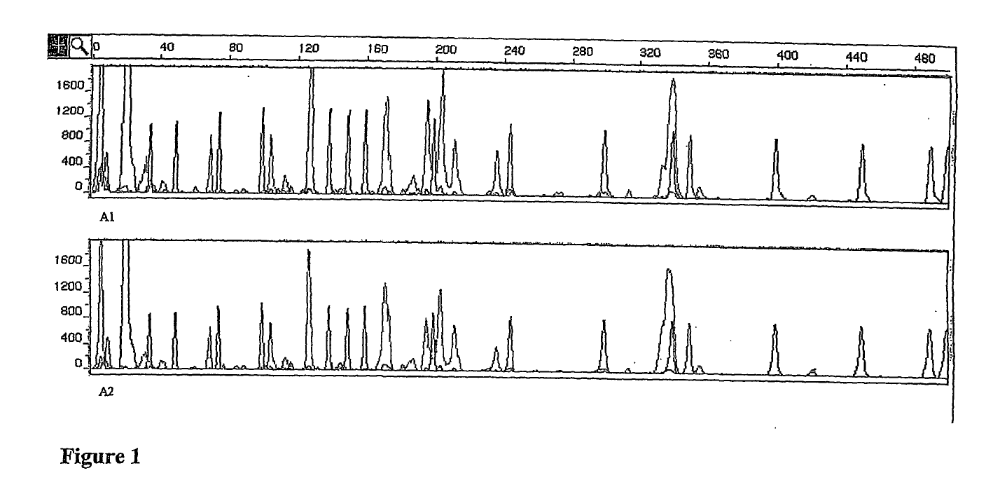

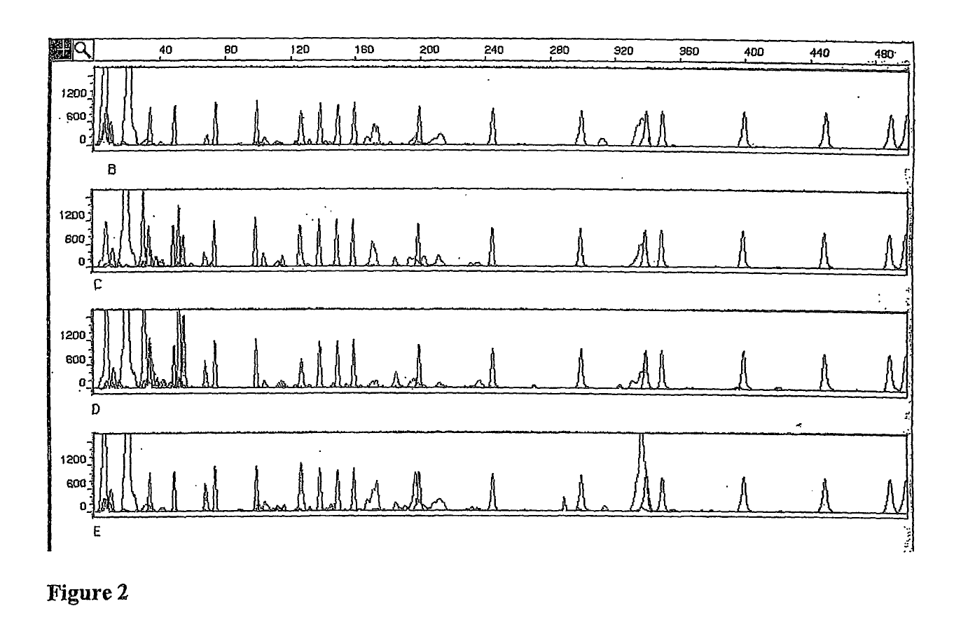

The microbial community profiles for the shoe print and soil collected from the suspect's shoes are shown in electropherograms of FIG. 1. The similarity index of these two profiles was suprisingly very high (>0.9 as per Sorenson's index), as evidenced by the fact that the electropherograms are almost entirely superimposable. FIG. 2 shows the electropherograms of the reference soils and Table 2 shows the similarity indexes of all profiles analysed. It can be seen that there are major differences between the profiles of the DNA from reference soils, the soil from the crime scene, and that soil from the suspect's shoes. In all cases the similarity index was less than 0.67.

TABLE 2
Similarity index for Forensic samples and reference soil samples

| Site | A1 | A2 | B | C | D | E |
|---|---|---|---|---|---|---|
| A1 | — | 0.91 | 0.54 | 0.59 | 0.56 | 0.48 |
| A2 | 0.91 | — | 0.53 | 0.67 | 0.64 | 0.54 |
| B | 0.54 | 0.53 | — | 0.63 | 0.59 | 0.57 |
| C | 0.59 | 0.67 | 0.63 | — | 0.73 | 0.62 |
| D | 0.56 | 0.64 | 0.59 | 0.73 | — | 0.50 |
| E | 0.48 | 0.54 | 0.57 | 0.62 | 0.50 | — |

Scenario 2

The microbial community profiles from the soil from the left knee impression in the soil, and the soil extracted from the left knee of the jeans were very similar with a Sorenson's index of 0.82. The similarity of the right knee soil impression in the water extract and TE buffer extract were also very high (0.78 and 1.00 respectively).

This example illustrates that a soil DNA profile may be obtained from a small sample of soil recovered from the sole of a shoe, that is, the sample size is likely to be encountered in forensic case work. It has also been shown that this profile is representative of the site of collection, and different from reference soil samples collected at other sites.

It will be appreciated that significant forensic evidence may be obtainable by the process described above.

It is also envisaged that a number of soil samples from known locations could be tested by way of the process described above. It will be appreciated that over time a database of soil sample profiles and indexed locations could be established.

It is also envisaged that a dental cast may be taken of a print, such as a shoe print at a crime scene. It is anticipated that a soil sample could be taken from the dental cast which may be suitable for use as the sample of known origin in the process defined above.

Wherein the foregoing description reference has been made to integers or components having known equivalents then such equivalents are herein incorporated as if individually set forth.

Although the invention has been described by way of example and with reference to possible embodiments thereof, it is to be appreciated that improvements and/or modifications may be made thereto without departing from the scope or spirit thereof.

### REF RENCES

Bergquist, P. and Saul, D. (1996). Microbial Diversity—Its unexplored territory. Microbiology Australia. 17, 22-24.

Borneman, J. and Triplett, E. W. (1997). Molecular microbial diversity in soils from Eastern Amazonia: evidence for unusual micro-organisms and microbial population shifts associated with deforestation. Applied and Environmental Microbiology. 63, 2647-2653.

Fournier, D., Lemieux, R. and Couillard, D. (1998). Genetic evidence for highly diversified bacterial populations in wastewater sludge during biological leaching of metals. Biotechnology Letters. 20, 27-31.

Fisher, M. M. and Triplett, E. W. (1999). Automated approach for ribosomal intergenic spacer analysis of microbial diversity and its application to freshwater bacteria communities. Applied and Environmental Microbiology. 65, 4630-4636.

Horrocks, M. and Walsh, K. A. J. 1998. Forensic palynology: assessing the value of the evidence. Review of Palaeobotany and Palynology, 103. 69-74.

Leeflang, P. and Smit, E. (1997). Use of Expand™ PCR system to amplify the 16S ribosomal genes for characterization of bacterial communities in soil. Biochemica. 1, 16-18.

Marumo, Y., Sugita, R. and Seta, S. (1995). Soil as evidence in criminal investigation. Identification Reference Centre, National Research Institute of Police Science, National Police Academy, Japan.

Moyer, C. L., Tiedje, J. M., Dobbs, F. C. and Karl, D. M. (1996). A computer-simulated restriction fragment length polymorphism analysis of bacterial small-subunit rRNA gene: Efficacy of selected tetrameric restriction enzymes for studies of microbial diversity in nature. Applied and Environmental Microbiology. 62, 2501-2507.

Osborne, A. M., Moore, E. R. B. and Timmis, K. N. (2000). An evaluation of terminal-restriction fragment length polymorphism (T-RPLF) analysis for the structure of microbial community structure and dynamics. Environmental Microbiology 2, 39-50.

Thornton, J. I. (1986). Forensic soil characterisation. Forensic Science Progress. 1, 3-35.

Ward, D. M., Bateson, M. M., Weller, R. and Ruff-Roberts, A. L. (1992). Ribosomal RNA analysis of microorganisms as they occur in nature. Advances in Microbial Ecology. (Eds Marshall, K. C.) Plenuim Press (New York), 219-286.

Yeates, C. (1996). Microbial Diversity. Microbiology Australia. 17, 229-231.

What we claim is:

1. A process suitable for determining the origin of one or more samples containing micro flora, the process including the steps of:
taking at least one sample of unknown origin containing micro flora, and at least one sample of known origin containing micro flora;
extracting the DNA from the micro flora of each sample;
amplifying the DNA of any part from each sample;
conducting an analyses of the amplified DNA for each sample; and
comparing the analysis of the amplified DNA from the sample of unknown origin with the sample of known origin.

2. A process according to claim 1, wherein the process is applied to a soil sample of unknown origin.

3. A process according to claim 1 or claim 2, wherein the DNA to be amplified includes both a conserved and variable region of a gene.

4. A process according to claim 3, wherein the DNA to be amplified includes at least a portion of the 16S rRNA gene.

5. A process according to claim 3, wherein the DNA to be amplified includes at least a portion of the 23S rRNA gene.

6. A process according to any one of claims 1 to 5, wherein the analysis of the amplified DNA is a polymorphic analysis.

7. A process according to claim 6, wherein the polymorphic analysis is a restriction fragment length polymorphism analysis.

8. A process according to claim 6 or claim 7, wherein the polymorphic analysis is performed on the 16S rRNA gene.

9. A process according to claim 6 or claim 7 wherein the polymorphic analysis is performed on the 235 rRNA gene.

10. A process according to any one of claims 6 to 9, wherein the polymorphic is performed by a visual comparison.

11. A process according to any one of claims 6 to 9, wherein the polymorphic comparison is performed by a mathematical process.

12. A process according to claim 11, wherein the mathematical polymorphic comparison is performed using a computer algorithm.

13. A process according to any one of claims 1 to 12, wherein the process is applied to forensic analysis.

14. A kit containing appropriate materials that are used so as to determine the origin of one or more samples containing microflora in accordance with any one of claims 1 to 13.
